SEQUENCE LISTING (4 sequences)

SEQ ID NO: 1 - DNA, 1792 bp, Homo sapiens
  human protooncogene protein HPP1
gcggcaggcgcggccatggcgcagctgggaaagctgctcaaggagcagaagtacgaccggcagctgaggttgtggggtgatcatgggcaagaggctttagaatctgctcatgtttgcctaataaatgcaacagccacaggaactgaaattcttaaaaacttggtactaccaggtattggttcgtttacaattattgatggaaatcaggtcagcggagaagatgctggaaacaatttcttccttcaaagaagcagtatcggcaagaaccgagctgaagctgccatggaattcttacaagaattaaatagcgatgtctctggaagttttgtggaagagagtccagaaaaccttctagacaatgatccctcatttttctgtaggtttactgttgtagttgcaactcagcttcctgaaagcacttcactacgcttagcagatgtcctctggaattcccagattcctcttttgatctgtaggacatatggactagttggttatatgaggatcattataaaagaacatccagtaatagaatctcatccagataatgcattagaggatctacgactagataagccatttcctgaactgagagaacattttcagtcctatgatttggatcatatggaaaaaaaggaccacagtcatactccatggattgtgatcatagctaaatatttagcacagtggtatagtgaaacaaatggacgaatacctaaaacgtataaagaaaaagaggacttcagagatttgattagacaaggaattctaaaaaatgaaaatggggctccagaagatgaagagaattttgaagaagctattaaaaatgtgaacacagcactaaatacaactcagatcccaagcagtattgaagatatatttaatgatgatcgctgcataaatatcaccaaacagactccatcattttggattttagctcgtgccttaaaggaatttgtggccaaagagggtcaaggaaatttacctgttcgaggcacaattcctgatatgattgcagattcaggcaaatatataaaactgcaaaacgtttaccgtgaaaaagcaaagaaagatgctgccgctgtgggtaatcatgttgccaaattgctgcagtccattggccaggcaccagagtccatttcagagaaagaattaaaattactctgcagcaattctgcatttcttcgagtggtaagatgtcgatccttagctgaagaatatggtttggatacaattaacaaggatgaaattatttctagcatggacaatccagataatgaaatagtgttgtacttaatgttacgggctgttgatagatttcataaacaacagggtagatatccaggagtatctaactatcaagttgaagaagatataggaaagttgaagtcttgtctcactggcttccttcaggaatatggtttatctgtaatggtgaaagatgattatgtccacgaattttgccgatatggagctgctgagccacataccattgctgcattcttggggggagctgctgctcaagaggtcatcaaaataatcaccaaacaatttgtaatttttaataatacttacatttacagtggcatgtcacaaacttcagcaactttccagttgtagagtaagcaagcaccttaagtagtgtgttaatgattgaaactgtaattgccttcgggttgtgctttagtctgtaaaattctaaaggagagctgctaaattgtttttttaataaacatttttttcatttgtaataaaaaaaaaaaaaaaaaaaaaaaaaaaaaaaaaaaaaaaa

SEQ ID NO: 2 - protein, 534 aa, Homo sapiens
  human protooncogene protein HPP1
MAQLGKLLKEQKYDRQLRLWGDHGQEALESAHVCLINATATGTEILKNLVLPGIGSFTIIDGNQVSGEDAGNNFFLQRSSIGKNRAEAAMEFLQELNSDVSGSFVEESPENLLDNDPSFFCRFTVVVATQLPESTSLRLADVLWNSQIPLLICRTYGLVGYMRIIIKEHPVIESHPDNALEDLRLDKPFPELREHFQSYDLDHMEKKDHSHTPWIVIIAKYLAQWYSETNGRIPKTYKEKEDFRDLIRQGILKNENGAPEDEENFEEAIKNVNTALNTTQIPSSIEDIFNDDRCINITKQTPSFWILARALKEFVAKEGQGNLPVRGTIPDMIADSGKYIKLQNVYREKAKKDAAAVGNHVAKLLQSIGQAPESISEKELKLLCSNSAFLRVVRCRSLAEEYGLDTINKDEIISSMDNPDNEIVLYLMLRAVDRFHKQQGRYPGVSNYQVEEDIGKLKSCLTGFLQEYGLSVMVKDDYVHEFCRYGAAEPHTIAAFLGGAAAQEVIKIITKQFVIFNNTYIYSGMSQTSATFQL

SEQ ID NO: 3 - DNA, 16 bp, Artificial Sequence
  Primer H-T11A
aagcttttttttttta

SEQ ID NO: 4 - DNA, 13 bp, Artificial Sequence
  Primer H-AP33
aagcttgctgctc

(19) United States
(12) Patent Application Publication
(10) Pub. No.: US 2006/0234967 A1
(43) Pub. Date: Oct. 19, 2006

(54) Human protooncogene and protein encoded therein

(76) Inventor: Jin Woo Kim, Seoul (KR)

Correspondence Address: JONES DAY, 222 EAST 41ST ST, NEW YORK, NY, 10017, US

(21) Appl. No.: 10/543,751

(22) PCT Filed: Jan. 27, 2004
(86) PCT No.: PCT/KR04/00137

§ 371 Date: Apr. 21, 2006

(30) Foreign Application Priority Data
Jan. 27, 2003 (KR) 10-2003-0005185

(51) Int. Cl. A61K 48/00; G01N 33/574; C07H 21/04; C12P 21/06; C12N 15/74; C12N 1/21

(52) U.S. Cl. 514/44; 435/7.23; 435/69.1; 435/252.33; 435/488; 530/350; 536/23.2

(57) ABSTRACT

The present invention relates to a novel proto-oncogene HPP1 (Human proto-oncogene 1), which is non-homologous to existing oncogenes and the protein encoded by the said oncogene. This novel proto-oncogene can be advantageously used in diagnosing of various cancers; in construction of transformed animals; and in anti-sense gene therapy. The present invention relates to a novel proto-oncogene HPP1 (Human proto-oncogene 1), which is non-homologous to existing oncogenes and the protein encoded by the said oncogene. This novel proto-oncogene can be advantageously used in diagnosing of various cancers; in construction of transformed animals; and in anti-sense gene therapy.

### FIELD OF THE INVENTION

The present invention relates to a novel proto-oncogene and protein encoded therein, and more particularly, to a human proto-oncogene (hereinafter “HPP1 proto-oncogene”) and a protein derived therefrom, which can be used in diagnosis of various cancers.

### BACKGROUND OF THE INVENTION

Higher animals including man each carry approximately 100,000 genes, but only about 15% thereof is expressed, and characteristics of individual's biological process, e.g., genesis, differentiation, homeostasis, responses to stimuli, control of cell segmentation, aging and apoptosis (programmed cell death), are determined depending on which genes are expressed (Liang, P. and A. B. Pardee, Science, 257: 967-971(1992)).

Pathogenic phenomena such as tumorigenesis are caused by gene mutation, which brings about changes in the mode of gene expression. Therefore, comparative studies of gene expressions in various cells have been conducted to provide bases for establishing viable approaches to the understanding of diverse biological phenomena.

For example, the mRNA differential display (DD) method suggested by Liang and Pardee is effective in elucidating the nature of tumor suppressor genes, cell cycle-related genes and transcriptional regulatory genes that control apoptosis (Liang, P. and A. B. Pardee supra). Further, the DD method has been widely used in examining the interrelationship of various genes in a cell.

It has been reported that tumorigenesis is caused by various genetic changes such as the loss of chromosomal heterozygosity, activation of oncogenes and inactivation of tumor suppressor genes, e.g., p53 gene (Bishop, J. M., Cell, 64: 235-248(1991); and Hunter, T., Cell, 64: 249-270(1991)). Further, it has been reported that 10 to 30% of human cancer arises from the activation of oncogene through amplification of proto-oncogenes.

Therefore, the activation of proto-oncogenes plays an important role in the etiology of many tumors and there has existed a need to identify proto-oncogenes.

The present inventor has endeavored to unravel the mechanism involved in the tumorigenesis of cervical cancer; and, has unexpectedly found that a novel protooncogene protein 1 (HPP1) is specifically over-expressed in cancer cells. This proto-oncogene can be advantageously used in diagnosis, prevention and treatment of various cancers, e.g., leukemia, lymphoma, colon, lung, skin, kidney, breast, colon, ovary, stomach, liver and uterine cervix cancers.

### SUMMARY OF THE INVENTION

Accordingly, the primary object of the present invention is to provide a novel proto-oncogene.

Other objects of the present invention are to provide:

A recombinant vector containing said proto-oncogene and a microorganism transformed therewith;

A protein encoded in said proto-oncogene;

A kit for diagnosis of cancer containing said proto-oncogene;

A kit for diagnosis of cancer containing said protein;

An anti-sense gene having a base sequence complementary to that of said proto-oncogene; and

A process for treating or preventing cancer and metastasis by using said anti-sense gene.

In accordance with one aspect of the present invention, there is provided a novel proto-oncogene having the nucleotide sequence of SEQ ID NO:.

In accordance with another aspect of the present invention, there is provided a recombinant vector containing said proto-oncogene and a microorganism transformed with said vector.

In accordance with still another aspect of the present invention, there is provided a protein having the amino acid sequence of SEQ ID NO: 2 derived from said proto-oncogene.

### BRIEF DESCRIPTION OF THE DRAWINGS

The above and other objects and features of the present invention will become apparent from the following description of the invention, when taken in conjugation with the accompanying drawings which respectively show:

FIG. 1: the result of DDRT-PCR, which verifies the manifestation of CA336 in CUMC-6 cancer cells, normal and tumor tissues of cervix, and metastatic tissues of lymph nodes.

FIG. 2A: the result of northern blot analysis, which verifies the manifestation of HPP1 proto-oncogene of the present invention in cervical cancer tissues.

FIG. 2B: the result obtained with the same sample of FIG. 2A hybridized with β-actin.

FIG. 3A: the result of Northern blot analysis for HPP1 proto-oncogene expressed in normal human 12-lane multiple tissues.

FIG. 3B: the results obtained with the same sample of FIG. 3A hybridized with β-actin.

FIG. 4A: the result of Northern blot analysis for HPP1 proto-oncogene expressed in human cancer cell lines.

FIG. 4B: the result obtained with the same sample of FIG. 4A hybridized with β-actin.

FIG. 5A: the result of Northern blot analysis for HPP1 proto-oncogene expressed in human tumor tissues of kidney, breast, colon, ovary, stomach and liver and their normal counterparts.

FIG. 5B: the result obtained with the same sample of FIG. 5A hybridized with β-actin.

FIG. 6: the sodium dodecyl sulfate (SDS)-PAGE results showing protein expression patterns and protein size before and after the IPTG induction of E. coli transformed with HPP1 proto-oncogene.

### DETAILED DESCRIPTION OF THE INVENTION

The novel proto-oncogene of the present invention, i.e., human protooncogene (HPP1), consists of 1792 base pairs and has the DNA sequence of SEQ ID NO: 1.

In SEQ ID NO: 1, the full open reading frame corresponding to base Nos. 16 to 1620 (1618-1620: termination codon) is a protein encoding region and the predicted amino acid sequence derived therefrom is shown in SEQ ID NO: 2 which consists of 534 amino acids (hereinafter “HPP1 protein”).

In consideration of the degeneracy of codons and the preferred codons in a specific organism wherein the proto-oncogene of the present invention is to be expressed, various changes and modifications of the DNA sequences of SEQ ID NO: 1 may be made, e.g., in the coding area thereof without adversely altering the amino acid sequence of the expressed protein, or in the non-coding area without adversely affecting the expression of the proto-oncogene. Therefore, the present invention also includes, in its scope, a polynucleotide having substantially the same base sequence as the inventive proto-oncogene, and a fragment thereof. As used herein, “substantially the same polynucleotide” refers to a polynucleotide whose base sequence shows 80% or more, preferably 90% or more, most preferably 95% or more homology to the proto-oncogene of the present invention.

The protein expressed from the proto-oncogene of the present invention consists of 534 amino acids and has the amino acid sequence of SEQ ID NO: 2. The molecular weight of this protein is about 60 kDa. However, various substitution, addition and/or deletion of the amino acid residues of protein may be performed without adversely affecting the protein's function. Further, a portion of the protein may be used when a specific purpose is to be fulfilled. These modified amino acid sequence and fragments thereof are also included in the scope of the present invention. Therefore, the present invention includes, in its scope, a polypeptide having substantially the same amino acid sequence as the protein derived from the oncogene of the present invention and a fragment thereof. As used herein, “substantially the same polypeptide” refers to a polypeptide whose amino acid sequence shows 80% or more, preferably 90% or more, most preferably 95% or more homology to the amino acid sequence of SEQ ID NO: 2.

The proto-oncogene HPP1, or the protein of the present invention can be obtained from human cancer tissues or synthesized using a conventional DNA or peptide synthesis method. Further, the gene thus prepared may be inserted to a conventional vector to obtain an expression vector, which may, in turn, be introduced into a suitable host, e.g., a microorganism such as an E. coli or yeast.

For example, E. coli DH5α was transfected with an expression vector comprising proto-oncogene HPP1, and E. coli DH5α/HCC-11/pCEV-LAC thus obtained was deposited with the Korean Collection for Type Cultures (KCTC) (Address: Korea Research Institute of Bioscience and Biotechnology (KRIBB), #52 Oun-dong, Yusong-ku, Taejon 305-333, Republic of Korea) on Dec. 5, 2002 under the accession number of KCTC 10397BP in accordance with the terms of Budapest Treaty on the International Recognition of the Deposit of Microorganism for the Purpose of Patent Procedure.

In preparing a vector, expression-control sequences, e.g., promoter, and terminator, etc., self-replication sequence and secretion signal, are suitably selected depending on the host cell used.

Through Northern blot analyses, the novel proto-oncogene HPP1 is not manifested in normal uterus tissues but it is manifested in uterine and cervical cancer tissues; therefore, HPP1 is believed to be a type of carcinogen. In addition to epithelial tissues such as cervical cancer tissue, the over expression of the proto-oncogene HPP1 of the present invention is also observed in cancers as leukemia, lymphoma, colon, lung, skin, kidney, breast, colon, ovary, stomach, liver and uterine cervix cancers. Therefore, the proto-oncogene of the present invention is believed to be a factor common to all forms of various cancer and it can be advantageously used in the diagnosis of cancers and the production of a transformed animal as well as in an anti-sense gene therapy.

A diagnostic method that can be performed using the proto-oncogene of the present invention may comprise, for example, the steps of hybridizing nucleic acids separated from the body fluid of a subject with a probe containing the proto-oncogene of the present invention or a fragment thereof, and determining whether the subject has the proto-oncogene by using a conventional detection method known in the art. The presence of the proto-oncogene HPP1 may be easily detected by labeling the probe with a radioisotope or an enzyme. Therefore, a cancer diagnostic kit containing the proto-oncogene of the present invention or a fragment thereof is also included in the scope of the present invention.

A transformed animal produced by introducing the proto-oncogene of the present invention into a mammal, e.g., mice, is also included in the scope of the present invention. In producing such a transformed animal, it is preferred to introduce the inventive proto-oncogene to a fertilized egg of an animal before the 8-cell stage. The transformed animal can be advantageously used in screening for carcinogens or anticancer agents such as chemotherapeutic drugs.

The present invention is also effective in gene therapy, and it also provides an anti-sense gene comprising an mRNA complimentary base sequence that is induced by the novel proto-oncogene HPP1.

The present invention also provides an anti-sense gene, which is useful in a gene therapy. As used herein, the term “anti-sense gene” means a polynucleotide comprising a base sequence which is fully or partially complementary to the sequence of the mRNA which is transcribed from the proto-oncogene HPP1 having the base sequence of SEQ ID NO: 1 or a fragment thereof, said nucleotide being capable of preventing the expression of the open reading frame (ORF) of the proto-oncogene by way of attaching itself to the protein-binding site of mRNA.

The present invention also includes within its scope a process for treating or preventing cancer in a subject by way of administering a therapeutically effective amount of the inventive anti-sense gene thereto.

In the inventive HPP1 anti-sense gene therapy, the anti-sense gene of the present invention is administered to a subject in a conventional manner to prevent the expression of the proto-oncogene. For example, the anti-sense oligodeoxynucleotide (ODN) is mixed with a hydrophobicized poly-L-lysine derivative by electrostatic interaction in accordance with the method disclosed by Kim, J. S. et al. (J. Controlled Release, 53: 175-182(1998)) and the resulting mixed anti-sense ODN is administered intravenously to a subject.

The present invention also includes within its scope an anti-cancer composition comprising the HPP1 anti-sense gene of the present invention as an active ingredient, in association with pharmaceutically acceptable carriers, excipients or other additives, if necessary. The pharmaceutical composition of the present invention is preferably formulated for administration by injection.

The amount of the HPP1 anti-sense gene actually administered should be determined in light of various relevant factors including the condition to be treated, the chosen route of administration, the age and weight of the individual patient, and the severity of the patient's symptoms.

The protein expressed from the inventive proto-oncogene HPP1 may be used in producing an antibody useful as a diagnostic tool. The antibody of the present invention may be prepared in the form of a monoclonal or polyclonal antibody in accordance with any of the methods well known in the art by using a protein having the amino acid sequence of SEQ ID NO: 2 or a fragment thereof. Cancer diagnosis may be carried out using any of the methods known in the art, e.g., enzyme linked immunosorbentassay (ELISA), radioimmunoassay (RIA), sandwich assay, immunohistochemical staining, western blot or immunoassay blot on polyacrylic gel, to assess whether the protein is expressed in the body fluid of the subject. Therefore, a cancer diagnostic kit containing the protein having the amino acid sequence of SEQ ID NO: 2 or a fragment thereof is also included in the scope of the present invention.

A continuously viable cancer cell line may be established by using the proto-oncogene of the present invention, and such a cell line may be obtained, for example, from tumor tissues formed on the back of a nude mouse by injecting fibroblast cells transformed with the proto-oncogene of the present invention. The cell lines thus prepared may be advantageously used in searching for anti-cancer agents.

The following examples are intended to further illustrate the present invention without limiting its scope.

### EXAMPLE 1

### Tumor Cell Culture and Isolation of the Total RNA

(Step 1) Tumor Cell Culture

Normal exocervical tissue specimens were obtained from uterine myoma patients during hysterectomy, and untreated primary cervical cancer and metastatic common iliac lymph node tissue specimens were obtained during radical hysterectomy. The human cervical cancer cell line is CUMC-6 (Kim J W et al., Gynecol Oncol, 62: 230-240 (1996)).

The cells obtained from the above-mentioned tissues and CUMC-6 cell line were cultured in Waymouth's MB 752/1 culture solution (Gibco, USA) containing 2 mM glutamine, 100 IU/ml penicillin, 100 μg/ml streptomycin, and 10% bovine fetal serum (Gibco, USA). The cells that show 95% viability while being dyed with trypan blue were used in this example (Freshney, “Culture of Animal Cells: A Manual of Basic Technique” 2^{nd} Ed., A. R. Liss New York, 1987).

(Step 2) Isolation of RNA and mRNA Differential Display

Total RNAs were extracted from the tissue specimens and cells from Step 1 using a commercial system (RNeasy total RNA kit, Qiagen Inc., Germany), and DNA contaminants were removed therefrom using Message clean kit (GenHunter Corp., Brookline, Mass.).

### EXAMPLE 2

### Differential Display Reverse Transcription, DDRT-PC

Differential display was conducted according to Liang and Pardee's RT-PCR (Science, 257: 967-971 (1992)) with minor modifications as follows.

0.2 μg each of the total RNAs obtained in Step 1 of Example 1 was subjected to reverse transcription using H-T11A primer of SEQ ID NO: 3 as an anchored oligo-dT primer (RNAimage kit, GenHunter, cor., MA, USA), followed by polymerase chain reaction (PCR) using the same anchored primer and the primer of SEQ ID NO: 4 (H-AP33 primer among RNAimage primer sets 1-5, H-AP 1-40) in the presence of 0.5 mM [α-³⁵S]-labeled dATP (1200 Ci/mmol). The PCR thermal cycle was repeated 40 times, each cycle being composed of: 95° C. for 40 sec., 40° C. for 2 min. and 72° C. for 40 sec., and the final extension reaction was carried out at 72° C. for 5 min. The PCR product thus obtained was subjected to electrophoresis in 6% polyacrylamide sequencing gels, followed by autoradiography to determine the location of bands expressed differentially.

The band of fragment CA336 cDNA of size 181 bp (nucleotide No. 1569-1749 of SEQ ID NO: 1) was excised from the dried sequencing gel and then heated for 15 min. to elute fragment CA336 cDNA. The fragment CA336 cDNA was amplified by conducting PCR under the same conditions except that [α-³⁵S]-labeled dATP and 20 μM dNTPs were omitted.

### EXAMPLE 3

### Cloning

The amplified fragment CA336 was cloned into pGEM-T Easy vector using the TA Cloning System (Promega, USA).

(Step 1) Ligation Reaction

2 μl of CA336 product obtained from Example 2, 1 μl of pGEM-T Easy vector (50 ng), 1 μl of T4 DNA ligase 10× buffer solution, and 1 μl of T4 DNA ligase (3 weiss unit/μl; T4 DNA ligase, Promega) were added into a 0.5 ml test tube. Then, distilled water was added to make the total volume of 10 μl and cultured at 14° C. overnight.

(Step 2) TA Cloning Transformation

TA cloning transformation was conducted as follows.

E. coli JM109 was cultured in 10 ml LB broth (Bacto-Trip 10 g, Bacto-yeast extract 5 g, NaCl 5 g) until its optical density reached approximately 0.3 to 0.6 at 600 nm. The culture mixture was kept in ice for about 10 minutes, then centrifuged at 4° C. for 10 minutes at 4000 rpm in order to isolate bacterial cells. The bacterial cells thus obtained were exposed in 10 ml of ice-cold 0.1 M CaCl₂ for 30 minutes to 1 hour to produce competent cells. The resultant mixture was centrifuged at 4° C., for 10 minutes at 4000 rpm. The cells are collected then suspended in 2 ml of ice-cold 0.1M CaCl_{2.}

200 μl of the competent cell suspension was placed in a new microfuge tube, then 2 μl of the ligation solution obtained in Step 1 was added thereto. The mixture was cultured in a 42° C. water bath for 90 seconds, and then, chilled quickly to 0° C. 800 μl of SOC medium (Bacto-Tripton 2.0 g, Bacto-yeast extract 0.5 g, 1M NaCl 1 ml, 1M KCl 0.25 ml, TDW 97 ml, 2 M Mg²⁺, 2 M Glucose 1 ml) was added thereto and the mixture was incubated at 37° C. in a rotary shaking incubator at 220 rpm for 45 minutes.

25 μl of X-gal (stored in 40 mg/ml dimethylformamide) was smeared onto ampicillin containing LB plate stored in a 37° C. incubator using a glass rod. Then 25 μl of the transformed cells were smeared onto the plate using a glass rod, and incubated overnight at 37° C. 3 to 4 colonies were selected from the plate and cultured on separate ampicillin-containing LB plates. In order to produce a plasmid, a transformed E. coli JM109/CA336 was selected and cultured on 10 ml of terrific broth (TDW 900 ml, Bacto-Trip 12 g, Bactor-yeast extract 24 g, glycerol 4 ml, 0.17 M KH₂PO₄, 0.72 M K₂HPO₄ 100 ml).

### EXAMPLE 4

### Separation of Recombinant Plasmid DNA

Using the Wizard™ Plus Minipreps DNA Purification Kit (Promega, USA), CA336 plasmid DNA was separated from the transformed E. coli.

The separated plasmid DNA was treated with EcoRI restriction enzyme, and subjected to 2% gel electrophoresis to confirm the insertion of CA336 sequence in the plasmid.

### EXAMPLE 5

### Analysis of DNA Base Sequence

The CA336 PCR product obtained in Example 2 was amplified using a known method, and cloned. The sequence of the amplified CA336 PCR fragment analyzed using the Sequenase version 2.0 DNA sequencing kit (United States Biochemical, Cleveland, Ohio, USA) according to the dideoxy chain termination method corresponded to nucleotide numbers 1569 to 1749 of SEQ ID NO: 1 and this DNA fragment was designated “CA336”.

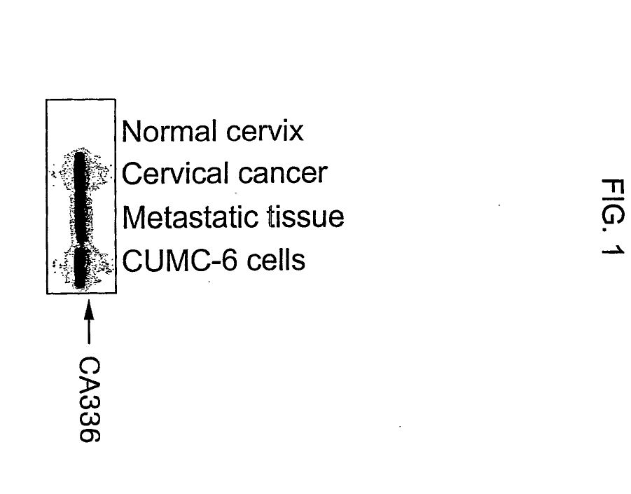

Using the 3′ H-T11A primer of SEQ ID NO: 3 and 5′ random primer H-AP33 of SEQ ID NO: 4, the cDNA fragment of 181 bp obtained above (CA336) was subjected to DDRT-PCR and then verified through electrophoresis. As illustrated in FIG. 1, the cDNA fragment (CA336) was expressed in metastasis lymph node tissues and CUMC-6 cells but not in normal tissues.

### EXAMPLE 6

### HPP1 Proto-Oncogene cDNA Library Screening

A bacteriophage λgt11 human lung embryonic fibroblast cDNA library (Miki, T. et. al., Gene, 83:137-146, 1989) was screened by plaque hybridization with ³²P-labeled CA336 cDNA probe. From the human lung embryonic fibroblast cDNA library, a full length HPP1 cDNA clone in vector pCEY-LAC was obtained. This vector was registered at GenBank, USA on May 5, 2001 (GenBank No.: AY197612; Open to public: May 1, 2004).

HPP1 has the 1792 bp sequence of SEQ ID NO: 1 and it is hypothesized that, in the base sequence of SEQ ID NO: 1, the entire open reading frame of the HPP1 proto-oncogene is believed to correspond to the nucleotide number 16 to 1620 which encodes the protein of SEQ ID NO: 2.

The HPP1 clone inserted in λ pCEV vector was cleaved with NotI to obtain an ampicillin resistant pCEV-LAC phagemid vector (Miki, T. et. al., Gene, 83:137-146, 1989).

The pCEV-LAC vector containing the HPP1 gene was ligated using T4 DNA ligase to produce HPP1 plasmid DNA, and then E. coli DH5α was transformed with the ligated clones.

The recombinant E. coli. DH5α/HCC-11/pCEV-LAC thus obtained was deposited with Korean Collection for Type Cultures, KCTC (Address: Korea Research Institute of Bioscience and Biotechnology (KRIBB), #52 Oun-dong, Yusong-ku, Taejon 305-333, Republic of Korea) on Dec. 5, 2002 under the accession number of KCTC 10397BP in accordance with the terms of Budapest Treaty on the International Recognition of the Deposit of Microorganism for the Purpose of Patent Procedure.

### EXAMPLE 7

### Northern Blot Analysis

Total RNAs were prepared from normal exocervical tissue, primary cervical cancer tissue; and human cervical cancer cell lines CaSki (ATCC CRL 1550) and CUMC-6, respectively, by repeating the procedure of Example 1. 20 μg each of the denatured total RNAs was electrophoresed through 1% formaldehyde agarose gel and transferred to a nylon membrane (Boehringer-Mannheim, Germany). The blots were hybridized with ³²P-labeled random-primed HPP1 cDNA probe which was prepared using a Rediprime II random prime labeling system (Amersham, England). The northern blot analysis was repeated twice and the result was quantified by densitometry. The blots were hybridized with a β-actin probe to confirm mRNA integrity.

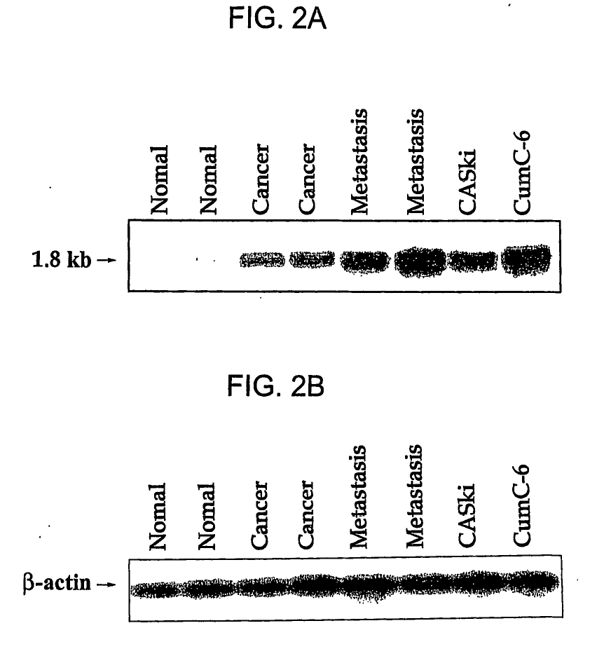

FIG. 2A shows the northern blot analysis results obtained for normal cervical tissues, primary cervical cancer tissues, primary cervical cancer metastasis lymph nodes tissues, and cervical cancer cell lines CUMC-6 and CaSki using the HPP1 cDNA probe; and FIG. 2B, the same blots, hybridized with a β-actin probe. As can be seen from FIG. 2A, the expression level of HPP1 gene was elevated in the cervical cancer tissues and the cervical cancer cell lines but nearly absent in all normal cervical tissues.

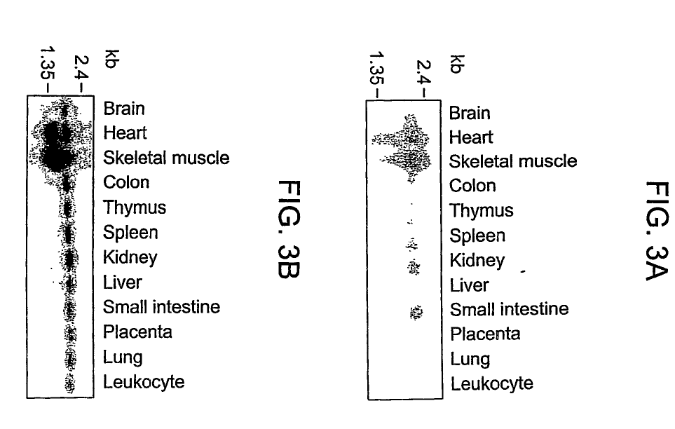

FIG. 3A shows the northern blot analysis results obtained for normal human tissues of brain, heart, skeletal muscle, colon, thymus, spleen, kidney, liver, small intestine, placenta, lung and peripheral blood leukocyte (Clontech), using HPP1 cDNA probe; and FIG. 3B, the same blot, hybridized with a β-actin probe. As can be seen in FIG. 3A, HPP1 mRNA (about 1.8 kb) is faint or nearly absent in normal tissues.

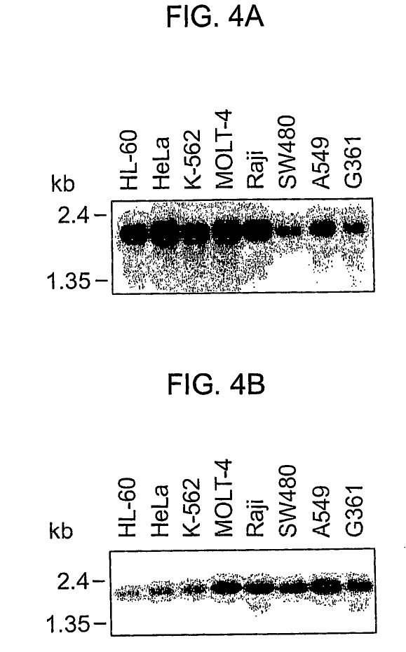

FIG. 4A shows the northern blot analysis results obtained for HPP1 proto-oncogene expressed in human cancer cell lines, HL-60, HeLa, K-562, MOLT-4, Raji cell, SW480, A549, and G361 (Clontech), and FIG. 4B, the blots hybridized with a β-actin probe to detect the existence of mRNA. As can be seen in FIG. 4A, HPP1 is overexpressed in SW480 colon cancer cell, lung cancer cell line A549, and skin cancer cell line G361, and transcribed especially at a high level in promyelocytic leukemia HL-60, HeLa uterine cancer cell line, chronic myelogenous leukemia K-562, lymphoblastic leukemia MOLT-4 and Burkitt's lymphoma Raji.

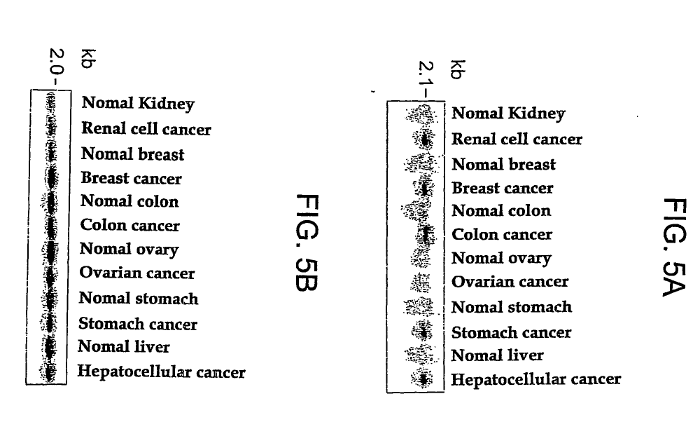

FIG. 5A shows the northern blot analysis results obtained for HPP1 proto-oncogene expressed in human tumor tissues of kidney, breast, colon, ovary, stomach and liver and their normal counterparts, and FIG. 5B, the blots hybridized with a β-actin probe to detect the existence of mRNA. the expression level of HPP1 gene was elevated in the cervical cancer tissues and the cervical cancer cell lines but nearly absent in all normal cervical tissues.

### EXAMPLE 8

### Determination of the Size of the Protein Expressed after the Transfection of E. coli with HPP1 Proto-Oncogene

A full-length HPP1 proto-oncogene of SEQ ID NO: 1 was inserted into BamHI/SalI restriction site in the multiple cloning site of pET-32b(+) vector (Novagen, Germany) and the resulting pET-32b(+)/HPP1 vector was transfected into E. coli BL21 (ATCC 47092). The expression protein, Trx•Tag, His•Tag and S•Tag are inserted at the front of the pET-32b(+) vector multiple cloning site. The transfected E. coli was incubated using an LB broth medium in a rotary shaking incubator, diluted by 1/100, and incubated for 3 hours. 1 mM isopropyl β-D-thiogalacto-pyranoside (IPTG, Sigma) was added thereto to induce the protein synthesis.

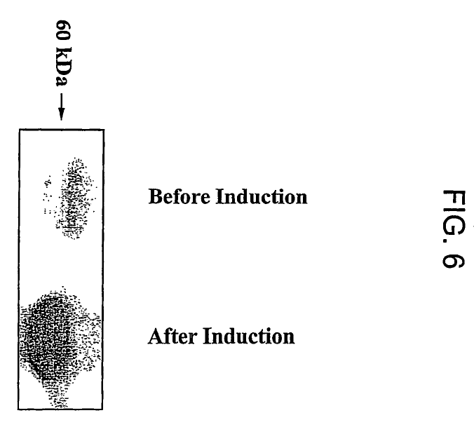

The E. coli cells in the culture were disrupted by sonication and subjected to gel electrophoresis using 12% sodium dodecyl sulfate (SDS) before and after the IPTG induction, respectively. FIG. 6 shows the SDS-PAGE results, which exhibit a protein expression pattern of the E. coli BL21 strain, transfected with pET-32b(+)/HPP1 vector. After the IPTG induction, a significant protein band was observed at about 80 kDa. This 80 kDa fused protein contained Trx•Tag, His•Tag and S•Tag of about 21 kDa and HPP1 protein of approximately 60 kDa.

While the invention has been described with respect to the above specific embodiments, it should be recognized that various modifications and changes may be made to the invention by those skilled in the art which also fall within the scope of the invention as defined by the appended claims.

What is claimed is:

1. A human proto-oncogene protein having the amino acid sequence of SEQ ID NO: 2.

2. A human proto-oncogene encoding the human proto-oncogene protein of claim 1.

3. The human proto-oncogene of claim 2, which contains a base sequence corresponding to base Nos. 16 to 1620 of SEQ ID NO: 1.

4. The human proto-oncogene of claim 2, which has a base sequence of SEQ ID NO: 1.

5. A vector comprising any of the proto-oncogene of claims 2 to 4.

6. A microorganism transformed with the vector of claim 5.

7. The microorganism of claim 6, which is E. coli DH5α/HCC-11/pCEV-LAC (Accession No.: KCTC 10397BP).

8. A process for preparing the protein of the claim 1 which comprises the step of culturing the microorganism of claim 6 or 7.

9. A kit for diagnosis of cancer, which comprises the protein of claim 1.

10. A kit for diagnosis of cancer, which comprises any of the proto-oncogene of claims 2 to 4.

11. An anti-sense gene having a base sequence complementary to the sequence of the mRNA transcribed from any of the proto-oncogene of claims 2 to 4 which is capable of binding the mRNA to inhibit the expression of the said proto-oncogene.

12. A composition for preventing or treating cancer and metastasis, which comprises a therapeutically effective amount of the anti-sense gene of claim 11 and a pharmaceutically acceptable carrier.
